(19) United States
(12) Patent Application Publication
(10) Pub. No.: US 2025/0197347 A1
(43) Pub. Date: Jun. 19, 2025

(54) BIS-TAURINE SALT AND ITS PREPARATION METHOD AND APPLICATION

(71) Applicant: ZHANGJIAGANG GREAT CHEMICAL CO., LTD., Suzhou (CN)

(72) Inventors: Yongqian Huang, Suzhou (CN); Hongjun Zhu, Suzhou (CN)

(21) Appl. No.: 18/926,345

(22) Filed: Oct. 25, 2024

(30) Foreign Application Priority Data
Dec. 13, 2023 (CN) 202311713571.5

(51) Int. Cl. C07C 303/32; C11D 1/28; C11D 3/00

(52) U.S. Cl. CPC C07C 303/32; C11D 1/28; C11D 3/0094

(57) ABSTRACT

Provided are a bis-taurine salt and its preparation method and application, which relate to the field of surfactant preparation. The preparation method is as follows: (1) reacting fatty acid and/or grease with an alkali metal salt of taurine to obtain a reaction product containing N-acyl taurine salt; and (2) directly reacting the reaction product obtained in step (1) with dimethyl carbonate without separation and purification to obtain N-acyl taurine salt and N-acyl methyl taurine salt. Also provided are the prepared bis-taurine salt and its application in preparation of high foaming surfactants. Compared with the prior art, the present disclosure does not use high-cost fatty acyl chloride and N-methyl taurine salt with potential safety and environmental hazards. Moreover, the bis-taurine salt of a desired mixing ratio is effectively and directly obtained through continuous reaction of a one-pot method, thus increasing reaction efficiency, avoiding caking, and improving usability of the bis-taurine salt.

### CROSS-REFERENCE TO RELATED APPLICATION

The present application claims priority from Chinese Patent Application No. 202311713571.5 filed on Dec. 13, 2023, the contents of which are incorporated herein by reference in their entirety.

### TECHNICAL FIELD

The present disclosure relates to the field of surfactant preparation, in particular to a bis-taurine salt and its preparation method and application.

### BACKGROUND

An N-acyl taurine salt surfactants have been widely used due to their extremely low irritation, non-toxicity and biodegradability. Such surfactants are good in compatibility with anionic, nonionic and amphoteric surfactants. They belong to an anionic surfactant with high safety, with excellent water solubility, hard water resistance, alkali resistance and acid resistance.

Typically, N-acyl taurine salt surfactants on the market are predominantly N-acyl methyl taurine salts. At present, the industrial production of N-acyl methyl taurine salts is mostly based on the Schotten-Baumann condensation process with acyl chloride as a main raw material, which involves an amidation reaction of fatty acyl chloride and N-methyl taurine salt in water/an organic solvent under the alkaline condition. There are two problems with this process:

1. With acyl chloride as a raw material, more chloride ions are introduced, and the subsequent desalination step will produce a large amount of chlorine-containing wastewater, which is environmentally unfriendly; acyl chloride is a dangerous material with poor safety; and moreover, the synthesis of acyl chloride from fatty acids increases costs significantly and thus is poor in economy.

2. A preparation method of sodium N-methyl taurate is as follows: first, reacting ethylene oxide with sodium bisulfite to produce sodium 2-hydroxyethyl sulfonate, and then reacting the obtained product with methylamine under the conditions of high temperature and high pressure to produce sodium N-methyl taurate. This method is carried out at the high temperature of 150-300° C. and under the high pressure of 10-25 Mpa, and thus the process conditions are harsh. Furthermore, this preparation method uses methylamine, which is a gas at room temperature, so that its raw material source, environmental protection, and labor protection are all problematic, and it has the disadvantages of being poor in environmental protection, safety, and economy.

In order to solve the problem of fatty acyl chloride, some researchers have tried a direct amidation condensation process with fatty acids as main raw materials, that is, fatty acids and sodium methyl taurate are dehydrated and condensed under the high temperature condition to synthesize the target product in one step. For example, the patent U.S. Pat. No. 5,496,959/U.S. Pat. No. 2,880,219/JP2002234868/U.S. Pat. No. 3,232,968/CN105175291/CN201510568940.5/CN115 160189, or the like describes a direct condensation process of fatty acids and sodium methyl taurate, but it also fails to solve the above-mentioned problems in the preparation of sodium N-methyl taurate.

The patent CN102875422 uses dimethyl sulfate to methylate sodium N-acyl taurate so as to generate sodium N-acyl methyl taurate, in which fatty acyl chloride is used. Additionally, the properties of dimethyl sulfate are similar to phosgene, making it a controlled reagent with poor safety and higher separation costs in the later stages.

In the previous study, the applicant of the present disclosure found that the “bis-taurine salt” mixed surfactant combination of N-acyl taurine salt and N-acyl methyl taurine salt had better performance than N-acyl taurine salt or N-acyl methyl taurine salt alone, and had applied for a patent. See the patent CN116098821A for details.

Although the “bis-taurine salt” surfactant can be prepared from the two separately produced N-acyl taurine salts through the aforementioned method, it inevitably has the problems of poor environmental protection, poor safety and poor economy at the same time.

Therefore, how to provide a simple, environmentally friendly, safe and economical method for preparing “bis-taurine salt” is one of the key issues studied by those skilled in the art.

### SUMMARY

The present disclosure provides a bis-taurine salt and its preparation method to address the problems of poor environmental friendliness, safety, and economy in the preparation process of N-acyl taurine salt and N-acyl methyl taurine salt in the prior art. According to the method provided by the present disclosure, firstly, fatty acid and taurine salt are directly subjected to amidation so as to synthesize N-acyl taurine salt, and then dimethyl carbonate is used to methylate the N-acyl taurine salt, so that “bis-taurine salt” formed by the N-acyl taurine salt and the N-acyl methyl taurine salt which are mixed in various proportions can be produced in one reactor according to the requirements. Furthermore, the applicant unexpectedly discovered during the study of the preparation method that the bis-taurine salt prepared using the preparation method of the present disclosure exhibited a significantly reduced dissolution temperature in a surfactant system.

In the present disclosure, “bis-taurine salt” refers to a combination of N-acyl taurine salt and N-acyl methyl taurine salt, which indicates the type of a compound rather than the compound itself. The bis-taurine salt specifically prepared by adopting the technical solution of the present disclosure shall prevail.

To achieve the above purpose, the technical solution adopted by the present disclosure is as follows:

A preparation method of bis-taurine salt includes the following steps:
(1) reacting fatty acid and/or grease with an alkali metal salt of taurine to obtain a reaction product containing N-acyl taurine salt; and
(2) directly reacting the reaction product obtained in step (1) with dimethyl carbonate without separation and purification to obtain N-acyl taurine salt and N-acyl methyl taurine salt.

Preferably, the reaction route of the step (1) is as follows:

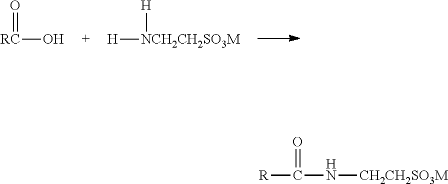

in the formula, R is a saturated or unsaturated hydrocarbon group of C7-C21; and M is selected from lithium ions, potassium ions, or sodium ions in alkali metal ions.

Preferably, the reaction route of the step (2) is as follows:

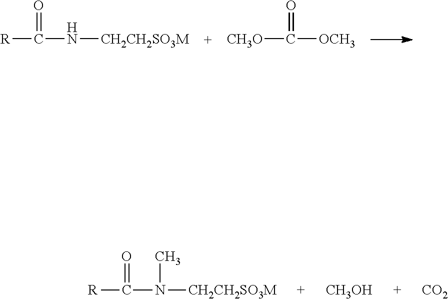

in the formula, R is a saturated or unsaturated hydrocarbon group of C7-C21; and M is selected from lithium ions, potassium ions, or sodium ions in alkali metal ions.

Preferably, the catalyst for the reaction in step (1) is composed of one or more of alkali metal or alkaline-earth metal hydroxides, alkali metal or alkaline-earth metal phosphates, alkali metal or alkaline-earth metal phosphites, alkali metal or alkaline-earth metal hypophosphites, alkali metal or alkaline-earth metal carbonates, alkali metal or alkaline-earth metal sulfates, alkali metal or alkaline-earth metal methoxy salts, alkali metal or alkaline-earth metal ethoxy salts, and alkali metal or alkaline-earth metal oxides.

Most preferably, the mass ratio of the catalyst to the fatty acid and/or grease in the reaction described in step (1) is 1:(200-330).

Preferably, the mass ratio of the fatty acid and/or grease to the alkali metal salt of taurine in step (1) is (2-5):(1-3).

Preferably, the fatty acid described in step (1) includes one or more selected from caprylic acid, capric acid, lauric acid, coconut acid, tridecylic acid, myristic acid, pentadecanoic acid, palmitic acid, heptadecanoic acid, stearic acid, isostearic acid, nonadecanoic acid, arachidic acid, behenic acid, alpha-undecylenic acid, oleic acid, linoleic acid, ricinoleic acid, and arachidic acid.

Further preferably, the fatty acid is selected from coconut acid and/or lauric acid.

Preferably, the grease described in step (1) is selected from natural vegetable oils and/or animal oil.

Further preferably, the vegetable oils include one or more selected from olive oil, castor oil, peanut oil, coconut oil, soybean oil, cottonseed oil, flaxseed oil, palm oil, and corn oil; and the animal oil is selected from beef tallow.

Most preferably, the vegetable oil is coconut oil.

Preferably, the alkali metals in the alkali metal salt described in step (1) include one or more selected from lithium, sodium, and potassium.

Further preferably, the alkali metal in the alkali metal salt described in step (1) is selected from sodium.

Preferably, reaction mediums used in the reaction described in step (1) include one or more selected from methanol, ethanol, isopropanol, acetone, ethyl acetate, propylene glycol, glycerol, ethylene glycol, and polyethylene glycol.

Further preferably, the reaction mediums include one or more selected from propylene glycol and glycerol.

Most preferably, the mass of the reaction medium in step (1) accounts for 0-99% of the total mass of all substances in the reaction system in step (1).

Preferably, the reaction conditions in step (1) are as follows: the temperature is 150-200° C., and the time is 2-8h.

Preferably, the reaction condition in step (2) is: a reflux reaction is carried out for 1-2h.

The present disclosure also provides the bis-taurine salt prepared by the above preparation method.

The present disclosure also provides application of the above bis-taurine salt in preparation of high foaming surfactants.

Compared with the prior art, the present disclosure has the following beneficial effects.

(1) The present disclosure directly performs an amidation reaction on fatty acid and/or grease and taurine salt, and then uses dimethyl carbonate for a methylation reaction, thus avoiding the use of high-cost fatty acyl chloride and N-methyl taurine salt with potential safety and environmental hazards. Moreover, the method provided by the present disclosure can effectively reduce the dissolution temperature of the prepared bis-taurine salt in a surfactant system where it is located, so that the surfactant system can maintain a liquid state within a larger temperature range, and the usability of the bis-taurine salt is improved. Furthermore, according to the present disclosure, the bis-taurine salt of a desired mixing ratio can be effectively and directly obtained through the continuous reaction of a one-pot method, so that the reaction efficiency is further increased, caking is avoided, and the production cost of the product is lowered.

(2) Meanwhile, the inventor unexpectedly found that the bis-taurine salt prepared by the present disclosure has better foaming property and milder cleaning power at a low dosage than the bis-taurine salt obtained by mixing regular commercially available products.

### DETAILED DESCRIPTION OF THE EMBODIMENTS

It should be noted that the raw materials used in the present disclosure are all common commercially available products, and their sources are not specifically limited.

### Example 1

200 g of lauric acid, 150 g of sodium taurate, 1 g of sodium hydroxide and 150 g of propylene glycol were put into a reaction flask equipped with a stirrer, a condenser and a feeding funnel. The mixture was heated up to 190° C. under nitrogen protection, and are kept heated and stirred for 2 hours; and the temperature was lowered to 110° C., and 100 g of dimethyl carbonate was added dropwise while stirring. After the dropwise addition was completed, reflux was maintained for 1 hour, and then the residual solvent was distilled off at 110° C. before the product was discharged.

The final product contained 329 g of sodium N-lauroyl methyl taurate and 15 g of sodium N-lauroyl taurate.

### Example 2

200 g of lauric acid, 300 g of sodium taurate, 1 g of sodium hydroxide and 150 g of propylene glycol were put into a reaction flask equipped with a stirrer, a condenser and a feeding funnel. The mixture was heated up to 190° C. under nitrogen protection, and are kept heated and stirred for 2 hours; and the temperature was lowered to 110° C., and 45 g of dimethyl carbonate was added dropwise while stirring. After the dropwise addition was completed, reflux was maintained for 1 hour, and then the residual solvent was distilled off at 110° C. before the product was discharged.

The final product contained 172 g of sodium N-lauroyl methyl taurate and 166 g of sodium N-lauroyl taurate.

### Example 3

330 g of coconut oil, 80 g of sodium taurate, 1 g of calcium oxide and 80 g of glycerol were put into a reaction flask equipped with a stirrer, a condenser and a feeding funnel. The mixture was heated up to 150° C. under nitrogen protection, and are kept heated and stirred for 7 hours; and 20 g of dimethyl carbonate was added dropwise while stirring. After the dropwise addition was completed, reflux was maintained for 2 hours, and then the residual solvent was distilled off before the product was discharged.

The final product contained 71 g of sodium N-cocoyl methyl taurate and 89 g of sodium N-cocoyl taurate.

### Example 4

400 g of coconut acid, 80 g of sodium taurate, 1 g of sodium hydroxide and 0.5 g of sodium hypophosphite were put into a reaction flask equipped with a stirrer, a condenser and a feeding funnel. The mixture was heated up to 200° C. under nitrogen protection, and are kept heated and stirred for 8 hours; and the temperature was lowered to 120° C., and 10 g of dimethyl carbonate was added dropwise while stirring. After the dropwise addition was completed, reflux was maintained for 1 hour, and then the residual solvent was distilled off before the product was discharged.

The final product contained 35 g of sodium N-cocoyl methyl taurate and 152 g of sodium N-cocoyl taurate.

### Example 5

300 g of coconut acid, 80 g of sodium taurate, 1 g of sodium hydroxide and 0.5 g of sodium hypophosphite were put into a reaction flask equipped with a stirrer, a condenser and a feeding funnel. The mixture was heated up to 200° C. under nitrogen protection, and are kept heated and stirred for 8 hours; and the temperature was lowered to 120° C., and 60 g of dimethyl carbonate was added dropwise while stirring. After the dropwise addition was completed, reflux was maintained for 1 hour, and then the residual solvent was distilled off before the product was discharged.

The final product contained 153 g of sodium N-cocoyl methyl taurate and 12 g of sodium N-cocoyl taurate.

### Effect Example 1: Test on Paste Performance of Bis-Taurine Salts Prepared in Examples 1-5 in the Preparation of Cleaning Compositions

The detection method used in this effect example is as follows:

Appearance (25° C.): A sample was placed at 25° C. and maintained at the consistent temperature for 48 hours, and whether the appearance is pasted or not was visually inspected.

Appearance (10° C.): A sample was placed at 10° C. and maintained at the consistent temperature for 48 hours, and whether the appearance is pasted or not was visually inspected.

Dissolution temperature: A sample was placed at −18° C. to form a paste, and then kept at 0° C. for 24 hours; and after that, the sample was heated up at a rate of 5° C./hour for observation of dissolution, and the temperature when the sample is completely dissolved was recorded as the dissolution temperature.

Paste spreadability at 25° C.: After a sample was placed at 25° C. and maintained at the consistent temperature for 48 hours, 1 g of the sample was extruded onto wet hands and rubbed for 10 times with the hands to observe whether it was completely spread; if there was particle residue, or blocky or soapy residue, it was proved to be unqualified; and if it was completely spread or completely dissolved, it was proved to be qualified.

In the effect example, the proportion of bis-taurine salt of each of the products prepared in Examples 1-5 was kept unchanged, and water was added into each of the products to prepare a surfactant system with a water content of 70%.

The preparation method was that: the product was added with a formula amount of water, heated up to 50° C. and stirred for dissolving, so that the surfactant system was obtained.

The performance of the surfactant system prepared by the above method was tested. The results are as follows:

| Surfactant | | | | | |
|---|---|---|---|---|---|
| system | Example 1 | Example 2 | Example 3 | Example 4 | Example 5 |
| Appearance | Transparent | Transparent | Transparent | Transparent | Transparent |
| (25° C.) | liquid | liquid | liquid | liquid | liquid |
| Appearance | White paste | Transparent | Transparent | White paste | White paste |
| (10° C.) | | liquid | liquid | | |
| Dissolution | 23.5 | 3.8 | 3.0 | 10.9 | 13.6 |
| temperature/° C. | | | | | |
| Paste spreadability | ∘ | ∘ | ∘ | ∘ | ∘ |
| at 25° C. | | | | | |

In the table, ∘ means that the sample passed the test on paste spreadability at 25° C.

By comparing the above results with Examples A1-9 in the patent application CN116098821A, it can be seen that the method provided by the present disclosure further reduces the dissolution temperature of the paste and improves the usability on the basis of the data of the above patent application, specifically:

The ratios of the bis-taurine salts (N-acyl taurine salt to N-acyl methyl taurine salt) in Examples 1-5 of the present disclosure are 15:329, 166:172, 89:71, 152:35 and 12:153, respectively.

However, the law of the data in Examples A1-9 in the patent application CN116098821A is that in the process of changing the ratio of bis-taurine salt (N-acyl taurine salt to N-acyl methyl taurine salt) from 9:1 to 1:9, the dissolution temperature of the paste shows a trend of first decreasing and then increasing, which is best at the ratio of 1:1, with the paste being dissolved at 5.3° C., and is worst at the ratios of 1:9 and 9:1, with the paste being dissolved at 19.1° C. and 33.3° C., respectively.

However, according to the examples of the present disclosure, it can be seen that the ratio of the bis-taurine salt in Example 1 is close to 1:22, which is far less than 1:9. It is unpredictable that the dissolution temperature of the product prepared by the method provided by Example 1 of the present disclosure is as low as 23.5° C., that is, in the case of the ratio of the bis-taurine salt that is not conducive to dissolution, a significantly reduced dissolution temperature is still obtained.

In Examples 2 and 3 of the present disclosure, a ratio of the bis-taurine salt close to 1:1 was used, and its dissolution temperature was further reduced to 3.8° C. and 3.0° C. compared to 5.3° C.; and the dissolution temperatures in Examples 4 and 5 were also as low as 10.9° C. and 13.6° C.

Moreover, the surfactant systems prepared in all the examples showed good paste spreadability at 25° C., benefiting from the improvement of the preparation process and the better ratio of the bis-taurine salt; and Examples 2 and 3 could also maintain a liquid state at 10° C.

In summary, the bis-taurine salt obtained by the preparation method provided by the present disclosure exhibits a significantly reduced dissolution temperature in the preparation of surfactant systems.

### Effect Example 2: User Experience Test of Bis-Taurine Salts Prepared in Examples 1-5 in the Preparation of Cleaning Compounds

The bis-taurine salts prepared in Examples 1-5 were subjected to a user experience test, and the specific test method was as follows:

A total of 20 trial users were recruited to evaluate the samples prepared in Examples 1-5 in turn. The evaluation method was that: a sample was placed at 25° C. and maintained at the consistent temperature for 48 hours, then 0.3 g of the sample was extruded onto wet hands and rubbed until the entire hands were covered with the sample, and the hands were washed with water. Evaluation forms were used to collect the trial users' opinions on the use of the samples, and the foaming ability during the washing process and the tightness after washing were evaluated. The results are as follows:

| Surfactant | | | | | |
|---|---|---|---|---|---|
| system | Example 1 | Example 2 | Example 3 | Example 4 | Example 5 |
| Foaming | ▴ | ▴ | ▴ | ▴ | ▴ |
| ability | | | | | |
| Tightness | ● | ● | ● | ● | ● |

The meanings of the symbols in the figures are:
▴: 14 or more trial users believe that the foam is rich;
Δ: 10-13 trial users believe that the foam is rich;
∇: 7-9 trial users believe that the foam is rich;
▾: 3-6 trial users believe that the foam is rich;
●: 14 or more trial users feel that there is no tightness after washing;
◯: 10-13 trial users feel that there is no tightness after washing;
□: 7-9 trial users feel that there is no tightness after washing; and
▪: 3-6 trial users feel that there is no tightness after washing.

Furthermore, as a comparison, the products including sodium N-lauroyl taurate (95%), sodium N-lauroyl methyl taurate (95%), sodium N-cocoyl methyl taurate (95%) and sodium N-cocoyl taurate (95%) produced by Zhangjiagang Great Chemical Company were also selected and respectively prepared, according to the same products and proportions as those in Examples 1-5, into: sodium N-cocoyl taurate and sodium N-cocoyl methyl taurate, or sodium lauroyl taurate and sodium lauroyl methyl taurate as controls for test. The test results are shown below (in the following table, the different control groups are abbreviated as sodium N-acyl taurine and sodium N-acyl methyl taurine):

| Content ratio of | | | | | |
|---|---|---|---|---|---|
| sodium N-acyl | | | | | |
| taurine and sodium | | | | | |
| N-acyl methyl | | | | | |
| taurine | 15:329 | 166:172 | 89:71 | 152:35 | 12:153 |
| Foaming ability | ∇ | ∇ | ∇ | ▾ | ∇ |
| Tightness | □ | □ | ▪ | □ | ▪ |

It can be seen that compared with the controls obtained by mixing commercially available products, Examples 1-5 prepared by the specific method provided by the present disclosure have significantly better foaming ability. Moreover, Examples 1-5 are significantly better than the controls in terms of tightness, which fully demonstrates that the cleaning composition prepared by the specific method of the present disclosure has better foaming ability and suitable cleaning power.

Finally, it should be noted that the above content is only used to illustrate the technical solution of the present disclosure, rather than to limit the scope of protection of the present disclosure. Simple modifications or equivalent substitutions made to the technical solution of the present disclosure by those of ordinary skill in the art do not depart from the essence and scope of the technical solution of the present disclosure.

What is claimed is:

1. A preparation method of bis-taurine salt, comprising the following steps:
(1) reacting fatty acid and/or grease with an alkali metal salt of taurine to obtain a reaction product containing N-acyl taurine salt; and
(2) directly reacting the reaction product obtained in step (1) with dimethyl carbonate without separation and purification to obtain N-acyl taurine salt and N-acyl methyl taurine salt.

2. The preparation method according to claim 1, wherein the reaction route of the step (1) is as follows: 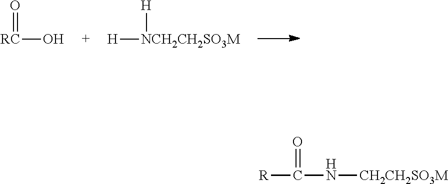
in the formula, R is a saturated or unsaturated hydrocarbon group of C7-C21; and M is selected from lithium ions, potassium ions, or sodium ions in alkali metal ions.

3. The preparation method according to claim 1, wherein the reaction route of the step (2) is as follows: 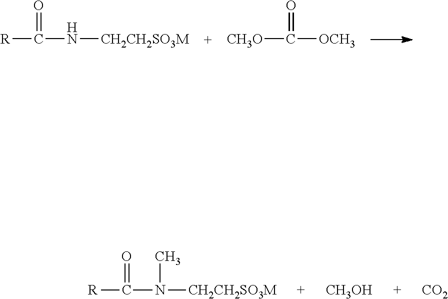
in the formula, R is a saturated or unsaturated hydrocarbon group of C7-C21; and M is selected from lithium ions, potassium ions, or sodium ions in alkali metal ions.

4. The preparation method according to claim 1, wherein the reaction described in step (1) is carried out in the presence of a catalyst, and the catalyst is composed of one or more of alkali metal or alkaline-earth metal hydroxides, alkali metal or alkaline-earth metal phosphates, alkali metal or alkaline-earth metal phosphites, alkali metal or alkaline-earth metal hypophosphites, alkali metal or alkaline-earth metal carbonates, alkali metal or alkaline-earth metal sulfates, alkali metal or alkaline-earth metal methoxy salts, alkali metal or alkaline-earth metal ethoxy salts, and alkali metal or alkaline-earth metal oxides.

5. The preparation method according to claim 4, wherein the mass ratio of the catalyst to the fatty acid and/or grease in the reaction described in step (1) is 1:(200-330); and the mass ratio of the fatty acid and/or grease to the alkali metal salt of taurine in step (1) is (2-5):(1-3).

6. The preparation method according to claim 1, wherein the fatty acid described in step (1) comprises one or more selected from caprylic acid, capric acid, lauric acid, coconut acid, tridecylic acid, myristic acid, pentadecanoic acid, palmitic acid, heptadecanoic acid, stearic acid, isostearic acid, nonadecanoic acid, arachidic acid, behenic acid, alpha-undecylenic acid, oleic acid, linoleic acid, ricinoleic acid, and arachidic acid.

7. The preparation method according to claim 6, wherein the fatty acid is selected from coconut acid and/or lauric acid.

8. The preparation method according to claim 1, wherein the grease described in step (1) is selected from natural vegetable oils and/or animal oil.

9. The preparation method according to claim 8, wherein the vegetable oils comprise one or more selected from olive oil, castor oil, peanut oil, coconut oil, soybean oil, cottonseed oil, flaxseed oil, palm oil, and corn oil; and the animal oil is selected from beef tallow.

10. The preparation method according to claim 1, wherein the alkali metals in the alkali metal salt described in step (1) comprise one or more selected from lithium, sodium, and potassium.

11. The preparation method according to claim 1, wherein reaction mediums used in the reaction described in step (1) comprise one or more selected from methanol, ethanol, isopropanol, acetone, ethyl acetate, propylene glycol, glycerol, ethylene glycol, and polyethylene glycol.

12. The preparation method according to claim 1, wherein the reaction conditions in step (1) are as follows: the temperature is 150-200° C., and the time is 2-8h.

13. The preparation method according to claim 1, wherein the reaction condition in step (2) is: a reflux reaction is carried out for 1-2h.

14. The bis-taurine salt prepared by the preparation method according to claim 1.

15. Application of the bis-taurine salt prepared by the preparation method according to claim 1 in preparation of high foaming surfactants.
